(19) United States
(12) Patent Application Publication
(10) Pub. No.: US 2011/0251555 A1
(43) Pub. Date: Oct. 13, 2011

(54) BALLOON-TIPPED ENDOSCOPIC SYSTEM WITH INVERTED SLEEVE

(75) Inventors: Richard W. Ducharme, Winston-Salem, NC (US); Tyler Evans McLawhorn, Winston-Salem, NC (US); Vihar C. Surti, Winston-Salem, NC (US)

(73) Assignee: Wilson-Cook Medical Inc., Winston-Salem, NC (US)

(21) Appl. No.: 12/903,595

(22) Filed: Oct. 13, 2010

Related U.S. Application Data
(60) Provisional application No. 61/252,981, filed on Oct. 19, 2009.

(51) Int. Cl. A61M 25/098; A61M 25/10

(52) U.S. Cl. 604/103.1; 604/96.01

(57) ABSTRACT

Multi-luminal endoscopic systems for sterilely delivering deployable devices. The system comprises an outer catheter comprising a distal portion and a wall that encloses an outer lumen; an inner catheter movably disposed within the outer lumen and having an inner lumen; a balloon-tipped catheter movably disposed within the inner lumen and having a distal portion and a proximal portion, wherein the distal portion of the balloon-tipped catheter comprises a balloon; a deployable device within the outer lumen; an invertible sleeve within the outer lumen with a first section attached to the distal portion of the outer catheter and a second section attached to a push mechanism that is proximal to the deployable device; and wherein the balloon is expandable to contact the invertible sleeve to provide a seal to prevent bodily fluids from entering the outer lumen.

### RELATED APPLICATION

The present application claims the benefit of U.S. Provisional Application No. 61/252,981, filed on Oct. 19, 2009, the entirety of which is hereby incorporated by reference.

### TECHNICAL FIELD

The present invention relates to ballooned-tipped endoscopic devices useful in natural orifice transluminal endoscopy surgery. The systems can be used to deploy therapeutic devices and obtain tissue samples

### BACKGROUND

Openings or perforations in the walls of internal organs and vessels may be naturally occurring, or formed intentionally or unintentionally. These openings may be used to gain access to adjacent structures of the body, such techniques being commonly referred to as transluminal procedures. For example, culdoscopy was developed over 70 years ago, and involves transvaginally accessing the peritoneal cavity by forming an opening in the cul de sac. This access to the peritoneal cavity allows medical professionals to visually inspect numerous anatomical structures, as well as perform various procedures such as biopsies or other operations, such as tubal ligation. Many transluminal procedures for gaining access to various body cavities using other bodily lumens have also been developed. One field of procedures has been referred to as Natural Orifice Transluminal Endoscopy Surgery (“NOTES”). Natural orifices such as the mouth, nose, ear, anus, or vagina may provide access to such bodily lumens and cavities. The bodily lumen(s) of the gastrointestinal tract are often endoscopically explored and can be utilized to provide access to the peritoneal cavity and other body cavities, all in a minimally invasive manner. U.S. Patent Publication No. 2008/0132948 discloses such a procedure and is incorporated herein by reference in its entirety.

Compared to traditional open surgery or laparoscopic surgery, transluminal procedures are less invasive by eliminating abdominal incisions (or other exterior incisions) and incision related complications, while also reducing postoperative recovery time, reducing pain, and improving cosmetic appearance. At the same time, there remain challenges to transluminal procedures, including providing a suitable conduit to the openings and body cavities, robust medical devices that are maneuverable via the conduit and operable within the body cavity, sterility of the conduit, maintaining insufflation of the body cavity, proper closure of the opening and prevention of infection. For example, when an opening is formed in a bodily wall of the gastrointestinal tract, such as in the stomach or intestines, spillage of the stomach contents, intestinal contents or other bodily fluids into the adjacent body cavity can occur. Travel of bacteria laden fluids outside of the gastrointestinal tract may cause unwanted and sometimes deadly infection.

One of the current challenges in NOTES procedures is sterile delivery of a material into the peritoneum and obtaining tissue samples in a sterile way.

### BRIEF SUMMARY

Herein provided is a multi-luminal system comprising an outer catheter comprising a distal portion and a wall that encloses an outer lumen; an inner catheter movably disposed within the outer lumen and having an inner lumen; a balloon-tipped catheter movably disposed within the inner lumen and having a distal portion and a proximal portion, wherein the distal portion of the balloon-tipped catheter comprises a balloon; a deployable device within the outer lumen. The system can also comprise an invertible sleeve within the outer lumen with a first section attached to the distal portion of the outer catheter and a second section attached to a push mechanism that is proximal to the deployable device. The balloon is expandable to contact the invertible sleeve to provide a seal to prevent fluids from entering the outer lumen.

The system can also comprise a push mechanism that is a push catheter having a lumen therethrough located within the outer lumen with a first position proximal to the balloon. The deployable device can be a medical device that provides a therapeutic treatment to an animal body. The deployable device is about the inner catheter and the inner catheter further comprises a push mechanism for deploying the deployable device.

Described herein is also a delivery system wherein the proximal portion of the balloon-tipped catheter is an elongated catheter shaft within the inner lumen and the balloon has a first predetermined diameter when inflated and a second predetermined diameter when deflated. The catheters can be concentric with the inner lumen of the inner catheter located within the lumen of the push catheter. The inner catheter and push catheter may not be concentric. The invertible sleeve can be comprised of biocompatible cloth or fabric mesh.

Also described herein is a method of delivery using a multi-luminal delivery system described herein. The method comprises introducing the system into an endoluminal vessel until the balloon reaches a desired location; deflating the balloon; placing the deflated balloon within the inner lumen of the inner catheter; manipulating the invertible sleeve such that the second section is distal to the first section; and deploying the deployable device by manipulating the outer catheter relative to the inner catheter such that the deployable device is distal to the outer catheter.

### BRIEF DESCRIPTION OF THE DRAWINGS

FIG. 1 is a cutaway perspective view of the delivery system with an inflated balloon and invertible sleeve.

FIG. 2 is a cutaway perspective view of the delivery system with a deflated balloon.

FIG. 3 is a perspective view of a hernia mesh being advanced by the push catheter and the invertible sleeve being deployed.

FIG. 4 is a cutaway perspective view of the delivery system with the hernia mesh deployed and the push catheter retracted into the system.

### DETAILED DESCRIPTION OF THE DRAWINGS AND THE PRESENTLY PREFERRED EMBODIMENTS

The term “prosthesis” means any replacement for a body part or for a function of that body part or any device that enhances or adds functionality to a physiological system.

The term “stent” means any device that provides rigidity, expansion force, or support to a prosthesis, such as a stent graft. In one configuration, the stent may represent a plurality of discontinuous devices. In another configuration, the stent may represent one device. Stents may have a wide variety of configurations and may be balloon-expandable or self-expanding. Typically, stents have a circular cross-section when fully expanded, so as to conform to the generally circular cross-section of a body lumen. In one example, a stent may comprise struts (elongate portions) and acute bends (curvilinear portions) that are arranged in a zigzag configuration in which the struts are set at angles to each other and are connected by the acute bends. Although an undulating configuration is used throughout this application, it is understood that the stent may have a sinusoidal or a zigzag configuration as well. One example of a stent configuration is a Z-stent. The stents as described in this disclosure may be attached to the exterior of the graft, the interior of the graft, and/or may be sandwiched between two or more layers of graft material.

A variety of biocompatible materials may be employed to construct the stent, or portions of the stent, including metals and/or alloys, medically acceptable polymers, and/or bioabsorbable polymers or materials. The metals and/or alloys may, among other things, include stainless steel, tantalum, nitinol, gold, silver, tungsten, platinum, inconel, cobalt-chromium alloys, and iridium, all of which are commercially available metals or alloys used in the fabrication of medical devices. In a preferred configuration, the stent is constructed from nitinol, stainless steel, and/or cobalt-chromium alloys.

The term “graft or graft material” means a generally cannular or tubular member which acts as an artificial vessel or prosthesis. A graft by itself or with the addition of other elements, such as structural components, can be an endoluminal prosthesis. The graft comprises a single material, a blend of materials, a weave, a laminate, or a composite of two or more materials.

The term “catheter” generally means medical devices including balloon-tipped catheters, guide catheters, and delivery catheters.

The term “deployable device” generally means a medical device that provides therapeutic treatment to a medically treatable area of an animal body. Deployable devices include, but are not limited to, a hernia mesh, ligating barrel, jejunal magnet, or stent graft.

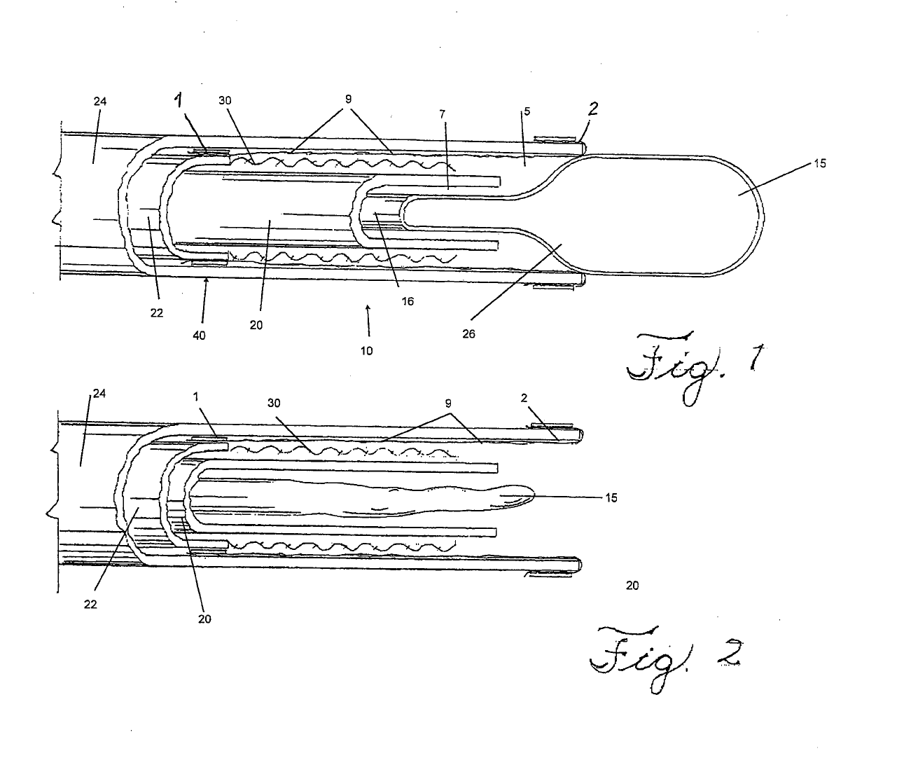

FIG. 1 shows an embodiment of multi-luminal delivery system 10 that minimizes the introduction of bacteria into the peritoneum and the risk of contaminating a sterile deployable device. The system is comprised of catheters that are roughly concentric and contain a deployable device that may be too large for placement in the access channel of an endoscope. The system can be introduced into the gastrointestinal tract through the mouth or other natural bodily orifice. FIG. 1 illustrates the delivery system 10 in the initial configuration suitable for introduction into the patient and advancement to the target site (i.e., where the deployable device is to be deployed).

As shown in FIG. 1, the system 10 comprises: an outer catheter 24, an inner catheter 20, a push catheter 22, a balloon-tipped catheter 26, and an invertible sleeve 9. The outer catheter 24 comprises a wall that encloses an outer lumen 5. The inner catheter 20 is movably disposed within the outer lumen 5 and has an inner lumen 7 that contains, at least partially, the balloon-tipped catheter 26. The distal portion of the inner catheter 20 can flare outwardly (not shown) to prevent the accumulation of potentially harmful bacteria from collecting on the outside of the inner catheter 20 and from contacting the sterile deployable device 30.

The invertible sleeve has a first section 2 that is attached to the distal portion of the outer catheter and a second section 1 that is attached to the push catheter 22. As shown in FIG. 1, the first section 2 of the invertible sleeve 9 is distal to the hernia mesh when the delivery device 10 is in the initial configuration. The distal portion of the balloon-tipped catheter 26 has a balloon 15 that contacts the invertible sleeve 9 near the first section 2. The seal provided by the contact of the balloon 15 and the invertible sleeve 9 helps to prevent bacteria or other potentially harmful elements from entering the outer lumen 5 or contaminating the deployable device 30. Although the embodiment illustrated in FIG. 1 comprises a balloon-tipped catheter 26 having an expandable balloon 15, the expandable member could be provided on the inner catheter 20 or on the invertible sleeve 9. So long as the expandable member is configured to form a seal sufficient to prevent contamination of the outer lumen 5.

Balloon-tipped catheters are manufactured in a variety of arrangements. The ballooned-tipped catheter 26 has a proximal elongated catheter shaft 16 that is movably disposed within the inner lumen 7 of the inner catheter 20. It is understood that the distal portion of the balloon-tipped catheter 26, the actual balloon 15, has a first larger diameter when inflated (FIG. 1) and a second smaller diameter when deflated (FIG. 2). As shown in FIG. 1, the inflated balloon 15 can contact the invertible sleeve 9 at the distal end of the outer catheter 24 such that blood and other bodily fluids are prevented from entering the outer lumen 5 and contacting the contents of the outer catheter as well as the deployable device 30. FIG. 2 shows the system with the balloon 15 deflated and retracted within the inner catheter 20.

The balloon-tipped catheter 26 can be made of materials capable of elastic expansion typically used in the field. For example, the balloon-tipped catheter 26 can comprise silicone, latex, or any other suitable material commonly used in the field. The balloon 15 can be tapered, bulbous, or cylindrical. The balloon-tipped catheter 26 can comprise a wire guide to assist in guiding the entire system 10 throughout the gastrointestinal tract. The balloon 15 can have a nipple like tip (not shown) to assist in advancing the catheter. Suitable alternative configurations for the balloon-tipped catheter 26 are disclosed in U.S. Provisional Application No. 61/141,568, the entire contents of which are hereby incorporated by reference.

As explained above, the invertible sleeve 9 assists in preventing contamination of the deployable device and the outer lumen. Without the invertible sleeve 9, the junction of the balloon 15 and the internal wall of the distal portion of the outer catheter 24 may come into contact with bodily fluids and bacteria as the system travels to a desired location. It may be possible that the deployable device may come into contact with remnants of these bodily fluids as it moves past the lip of the outer catheter 24. As shown in FIG. 1, the invertible sleeve 9 contacts the balloon 15 and is between the balloon 15 and the internal wall of the catheter 24. The first or distal section 2 of the invertible sleeve 9 is attached to the distal portion of the outer catheter 24 while the second or proximal section 1 of the invertible sleeve 9 is attached to the push catheter 22 at a point proximal to the deployable device 30. Alternatively, the proximal section 1 may be attached to the inner catheter 20. For example, the proximal section 1 of the invertible sleeve 9 may be attached to a push mechanism disposed on or about the inner catheter 20 proximal to the deployable device 30. The proximal section 1 may also be left unattached.

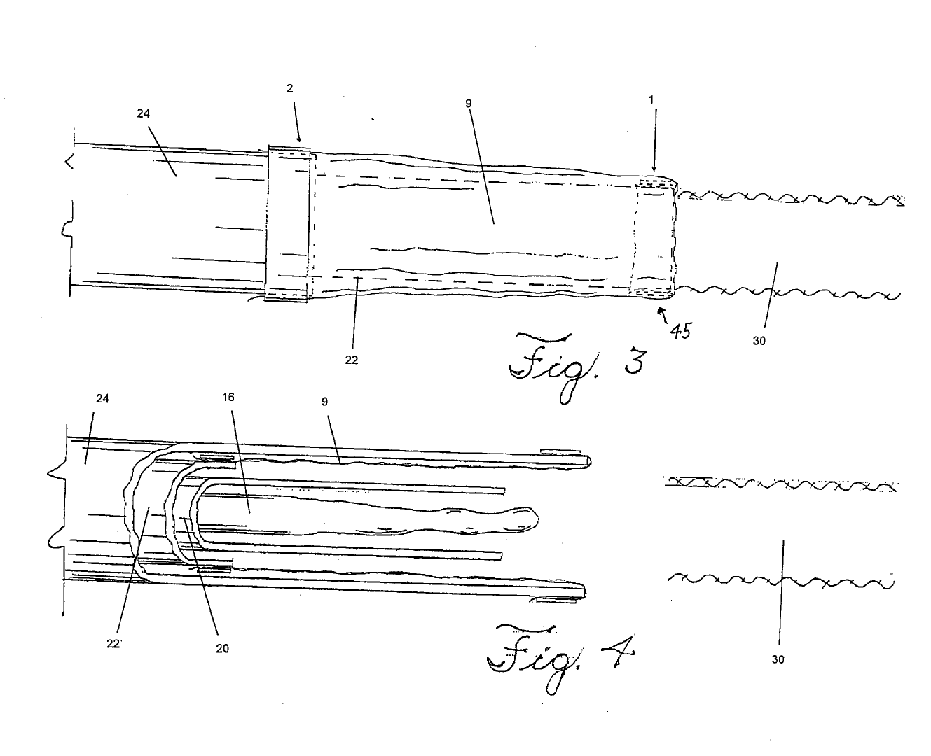

With any of the above configurations, any bacteria will come into contact with the inwardly facing side of the invertible sleeve 9. When the deployable device 30 is deployed as shown in FIGS. 3 and 4, the invertible sleeve 9 inverts its position such that the distal section 2 of the invertible sleeve 9 becomes proximal to the proximal section 1 of the invertible sleeve 9 and the side of the invertible sleeve 9 that was once inwardly facing becomes outwardly facing. The push catheter is shown in FIG. 3 with dashed lines 22. As a result, the deployable device 30 can be spared bacterial contact while being implanted.

The push catheter 22 is within the outer lumen and generally has the same diameter as the deployable device 30. The deployable device shown in the figures is a hernia mesh 30. The push catheter 22 is shown in a first position in FIG. 1. This first position 40 is proximal to the balloon 15. The first position 40 is generally the initial position of the push catheter 22 when the system 10 is inserted into the human body. The push catheter 22 is in the first position 40 when the deployable device 30 is not yet delivered or advanced/deployed out of the outer catheter 24 and the balloon 15 is still expanded and in contact with the wall of the outer catheter 24. FIG. 2 shows the balloon 15 deflated and withdrawn into the inner catheter 20. The push catheter's 22 second position 45 is obtained when it is advanced to deliver the deployable device 30, as shown in FIG. 3. Here, the second position 45 is distal to the balloon 15 and the outer catheter 24 such that the hernia mesh 30 is completely clear of the outer catheter 30. The distal end of the inner catheter 20 can be aligned with the distal end of the outer catheter 24 so as to not obstruct the delivery of the delivery device 30 as it is pushed by the push catheter 22 in a distal direction. Alternatively, the deployable device 30 may be delivered by retracting the outer catheter 24 while holding the pusher catheter 22 and the inner catheter 20 stationary.

The proximal elongated shaft 16 of the ballooned-tipped catheter 26 is disposed within the inner lumen 7 of the inner catheter 20. Once the delivery device 10 is advanced to the target site with the patient and the balloon 15 is deflated, it is retracted into the inner lumen 7. While within the inner catheter 20, the balloon 15 and any other bodily fluids disposed thereon are maintained separate from the deployable device 30. Contact between the deployable device 30 and the balloon 15 is minimized with the aid of the invertible sleeve 9 so as to maintain the sterility of the deployable device 30 until implantation. The seal provided by the balloon 15 minimizes the possibility of transferring bacteria or other microorganisms that may be considered harmful from a first environment into a second environment. The system 10 travels through the colon and out of an incision into the peritoneum. The system 10 may be exposed to fluids and bacteria in both environments. The seal helps prevent material found in the colon from entering the outer lumen 5 and being transferred to a second environment, such as the peritoneum. Once the balloon 15 is withdrawn inside the inner catheter 20, the push catheter 22 can be advanced distally to deploy the deployable device, the hernia mesh 30, out of the outer catheter 24. Alternatively, the outer catheter 24 can be retracted to deploy the deployable device 30.

The deployable device 30 and the push catheter 22 have smaller diameters than the outer catheter 24 but larger diameters than the inner catheter 20. Although a hernia mesh 30 is shown in these figures as the deployable device, other devices can be delivered using this system. For example, the delivery system 10 can also be used to deliver, for example, gauzes of any type, large volumes of fluid or powders, specimen retrieval bags, or slings. The deployable device can be a stent graft, ligating bands, or jejunal magnets. The deployable device can be any device used in endoscopy but is too large to fit in the accessory channel of an endoscope. The deployable devices may require other accoutrements for delivery. For instance, if actuating wires are needed for delivery of a stent graft they can be carried within the push catheter 22. Similarly, activation lines may be carried within the push catheter 22 for delivering ligating bands.

The system 10 can also comprise a wire guide to assist in delivery, such as is disclosed in U.S. Patent Publication No. 2010/0168612, the entire contents of which are hereby incorporated by reference. The system can also be adapted to accommodate joystick manipulation.

In an alternative embodiment, the push catheter 22 and the inner catheter 20 comprise a unitary construction. Or the push catheter 22 may be eliminated from the delivery system 10 altogether. With respect to the latter configuration, the function of the push catheter 22 may be replaced by a push mechanism disposed on the inner catheter 20 as described below.

This inner catheter 20 may comprise a push mechanism to assist in deploying the deployable device 30. The push mechanism can be a ridge having a diameter slightly larger than the diameter of the inner catheter 20 that proximally abuts the proximal end of a deployable device 30. The ridge can be used to advance the deployable device 30 out of the outer lumen 5 or hold the deployable device 30 in place while the outer catheter 24 is retracted. The ridge can be radiopaque. The balloon tip 15 can also comprise a wire guide to assist in placing the system 10.

The system is used to deliver deployable devices through a natural bodily orifice, such as the mouth, nose, or anus, with the balloon inflated to seal off bodily fluids. Through the mouth, the system would be able to access the upper gastrointestinal tract, the stomach, the duodenum, and the small intestine. Through the anus, the system could access the colon, which includes the large and small intestine. A system having comparable diameter can access the sinuses through the nose. Other areas of the body can be accessed with internal incisions in the stomach, vagina, bladder, or colon to perform procedures such as appendectomies, gastric revisions, ligation, or biopsies.

The system 10 is introduced into a human anus and advanced through the rectum and into the colon until the balloon tip 15 reaches a desired location. The system can be introduced transrectally, transvaginally, or transgastrically. The balloon tip 15 provides a seal to prevent bodily fluids from entering the outer lumen and contaminating the sterile deployable device. Because of its flexibility and contour, the balloon-tipped catheter 26 acts as a flexible tip to the system 10 that prevents damage to the anatomy as the system 10 winds its way through the gastrointestinal tract to the desired location.

Additional details of the components of the delivery system 10, and exemplary methods of using the system to deliver deployable devices, are disclosed in U.S. Patent Publication No. 2010/0168612, the entire contents of which are hereby incorporated by reference.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A multi-luminal delivery system comprising:
an outer catheter comprising a distal portion and a wall that encloses an outer lumen;
an inner catheter movably disposed within the outer lumen and having an inner lumen;
a balloon-tipped catheter movably disposed within the inner lumen and having a distal portion and a proximal portion, wherein the distal portion of the balloon-tipped catheter comprises a balloon;
a deployable device within the outer lumen; and
an invertible sleeve removably disposed within the outer lumen and having a first section attached to the distal portion of the outer catheter and a second section disposed proximal to the deployable device, the sleeve being disposed about the deployable device,
wherein the balloon is expandable to contact the invertible sleeve to provide a seal to prevent bodily fluids from entering the outer lumen.

2. The delivery system of claim 1 further comprising a push mechanism disposed proximally of the deployable device and configured to engage the deployable device during deployment.

3. The delivery system of claim 2 wherein the push mechanism comprises a push catheter movably disposed within the outer lumen and positioned with a first position proximal to the balloon.

4. The delivery system of claim 3 wherein the inner catheter and the outer catheter are concentric, and wherein the inner catheter is disposed within a lumen of the push catheter.

5. The delivery system of claim 3 wherein the inner catheter and push catheter are not concentric.

6. The delivery system of claim 3 wherein the second section of the sleeve is attached to the push catheter.

7. The delivery system of claim 2 wherein the push mechanism comprises a ridge disposed about the inner catheter and positioned proximal to the balloon.

8. The delivery system of claim 5 wherein the second section of the sleeve is attached to the inner catheter.

9. The delivery system of claim 1 wherein the deployable device is a medical device that provides a therapeutic treatment to an animal body.

10. The delivery system of claim 1 wherein the deployable device is disposed about the inner catheter, and wherein the inner catheter further comprises a push mechanism for engaging the deployable device.

11. The delivery system of claim 1 wherein the proximal portion of the balloon-tipped catheter is an elongated catheter shaft movably disposed within the inner lumen, and wherein the balloon has a first predetermined diameter when inflated and a second predetermined diameter when deflated, the first predetermined diameter being sufficient to contact and form a seal with the sleeve.

12. The delivery system of claim 1 wherein the invertible sleeve is comprised of biocompatible cloth or fabric mesh.

13. The delivery system of claim 1 wherein the push mechanism comprises radiopaque markers.

14. A method of delivery using a multi-luminal delivery system, the system comprising:
an outer catheter comprising a wall that encloses an outer lumen;
an inner catheter movably disposed within the outer lumen and having an inner lumen;
a balloon-tipped catheter movably disposed within the inner lumen having a distal portion comprising a balloon and a proximal portion;
the deployable device located with the outer lumen and about the inner catheter; and
an invertible sleeve within the outer lumen with a first section attached to the distal portion of the outer catheter and a second section disposed proximal to the deployable device, the sleeve being disposed about the deployable device;
said method comprising:
inflating the balloon so as to contact and form a seal with the invertible sleeve;
introducing the system into an endoluminal vessel until the system reaches a desired location;
deflating the balloon;
retracting the deflated balloon within the inner lumen of the inner catheter; and
deploying the deployable device by manipulating the outer catheter relative to the inner catheter such that the deployable device is distal to the outer catheter, wherein the step of deploying the deployable device includes the step of inverting the sleeve from the outer lumen such that the second section is distal to the first section.

15. The method of claim 14 wherein the delivery system further comprises a push mechanism for delivering the deployable device, and the step of deploying the deployable device further includes the step of engaging the deployable device with the push mechanism.

16. The method of claim 14 wherein the endoluminal vessel is the gastrointestinal tract.

17. The method of claim 14 wherein the deployable device is a medical device that provides a therapeutic treatment to an animal body.

18. The method of claim 14 wherein the deployable device is disposed about the inner catheter, and wherein the inner catheter further comprises a push mechanism for deploying the deployable device, the push mechanism comprising a ridge disposed about the inner catheter, and the step of deploying the deployable device further includes the step of engaging the deployable device with the ridge.

19. The method of claim 14 further comprising the step of introducing the system through the mouth of an animal.

20. The method of claim 14 wherein the invertible sleeve is comprised of biocompatible cloth or fabric mesh.

21. The method of claim 14 wherein the push mechanism comprises a radiopaque marker, and wherein the method further comprises the step of using fluoroscopy to monitor the position of the radiopaque marker during the steps of introducing and deploying.
